(19) United States
(12) Patent Application Publication
(10) Pub. No.: US 2020/0030375 A1
(43) Pub. Date: Jan. 30, 2020

(54) THERAPY AND METHODS OF INTRODUCING IMMATURE DENDRITIC CELLS AND/OR CYTOXIC T LYMPHOCYTE AND ANTI PD-1 / PD-L1 ANTIBODY FOR TREATMENT OF TUMORS

(71) Applicant: KENICHIRO HASUMI, TOKYO (JP)

(72) Inventor: KENICHIRO HASUMI, SUGINAMI-KU TOKYO (JP)

(73) Assignee: KENICHIRO HASUMI, TOKYO (JP)

(21) Appl. No.: 16/404,091

(22) Filed: May 6, 2019

Related U.S. Application Data
(63) Continuation-in-part of application No. 15/889,584, filed on Feb. 6, 2018, now Pat. No. 10,278,992.
Application No. 15/889,584 is a division of application No. 14/549,706, filed on Nov. 21, 2014, now Pat. No. 9,907,819.
Application No. 14/549,706 is a continuation-in-part of application No. 13/928,844, filed on Jun. 27, 2013, now Pat. No. 8,961,957.
(60) Provisional application No. 61/907,053, filed on Nov. 21, 2013.
(60) Provisional application No. 61/664,998, filed on Jun. 27, 2012.

(51) Int. Cl. A61K 35/15; A61K 39/395; C12N 5/0784; C12N 5/0783; A61K 35/17; C07K 16/24; A61K 38/19

(52) U.S. Cl. CPC A61K 35/15; A61K 39/3955; C12N 5/0639; C12N 5/0638; A61K 2039/5154; C07K 16/241; A61K 39/39558; A61K 38/19; A61K 35/17

(57) ABSTRACT

The invention relates to therapy and methods of applying the therapy to a patient. The invention includes the introduction of immature dendritic cells into the patient and the introduction of PD-1 and/or PD-L1 inhibitor into the patient. The immature dendritic cells are introduced intratumorally and/or through vessel and the PD-1 and/or PD-L1 inhibitor is introduced intratumorally and/or through vessel and/or subcutaneously. The immature dendritic cells can be formed by collecting monocyte cells from the patient and culturing the cells in a culture medium. The invention can be effective to regress, reduce or eliminate tumor cells in tumor tissue of the patients, including metastasized tumors. Further, the treatment of the invention is effective in the absence of conventional therapy, such as radiotherapy and chemotherapy.

### CROSS-REFERENCE TO RELATED APPLICATIONS

This continuation-in-part patent application claims priority from U.S. patent application Ser. No. 15/889,584, filed on Feb. 6, 2018, which is a divisional of U.S. patent application Ser. No. 14/549,706, filed on Nov. 21, 2014, now U.S. Pat. No. 9,907,819, issued on Mar. 16, 2018, both entitled “Therapy and Methods of Introducing Immature Dendritic Cells and/or Cytotoxic T Lymphocyte and Anti-TNF Antibody for Treatment of Tumors”, which claims priority from Provisional Application 61/907,053, also entitled “Therapy and Methods of Introducing Immature Dendritic Cells and/or Cytotoxic T Lymphocyte and Anti-TNF Antibody for Treatment of Tumors” filed on Nov. 21, 2013, and which is a continuation-in-part of U.S. patent application Ser. No. 13/928,844, now U.S. Pat. No. 8,961,957, issued on Feb. 8, 2015, which claims priority from Provisional Application 61/664,998, both entitled “Therapy and Method for Intratumorally Introducing Cytotoxic T Lymphocyte and/or NKT Cell With Anti-TNF and/or Anti-IL-10” filed on Jun. 27, 2013 and Jun. 27, 2012, respectively. The contents of which are incorporated herein.

### FIELD OF THE INVENTION

The invention relates to tumor cell and tumor tissue therapies, and methods for administering the therapies to treat cancer cells in a patient. The invention includes introducing intratumorally and/or through vessel immature dendritic cells and/or cytotoxic T lymphocytes (CTLs), and introducing intratumorally and/or through vessel and/or subcutaneously PD-1 and/or PD-L1 inhibitors to a cancer patient. The tumor therapies and methods of the invention are effective to treat the cancer patient in the presence or absence of conventional therapies, such as chemotherapy and/or radiotherapy.

### DESCRIPTION OF THE PRIOR ART

Cytotoxic T lymphocytes (CTLs) are an important component of cellular immunity. They play a critical role in the control of many infections and cancers. These T cells are responsible for “hunting down” other cells of the body that are infected by viruses or contain cancer, and destroying them. For example, when a virus or cancer is using a cell to reproduce, the cell displays some of the viral proteins or cancer components on its surface. The cytotoxic T cells can recognize these proteins or components and hone-in to destroy the infected or cancer-containing cells before they release the infection or cancer into the bloodstream. Many vaccines are effective, at least in part, by stimulating this type of T cell activation or response. CTLs can also create chemicals known as cytokines which assist in coordinating how the immune system fights against disease.

PD-1 is a protein that is present on CTLs. PD-L1 is present on cancer, e.g., tumor, cells. PD-1 and PD-L1 are checkpoint proteins that help keep the body's immune system in check. Since an important part of the immune system is its ability to distinguish between normal cells in the body and those that are “foreign” as well as attack the foreign cells, checkpoints provide a means for activating or inactivating the immune system to start or halt, respectively, an immune response.

PD-1 normally acts as a type of “off switch” or “brake” that blocks or halts CTLs from attacking other cells including cancer cells. Therefore, the association or interaction of PD-1 on CTLs and PD-L1 on cancer, e.g., tumor, cells decreases the ability of the CTLs to attack and kill the cancer cells. As a result of binding PD-1 and PD-L1, cancer cells can evade immune attack, and allows them to survive and grow.

PD-1 and/or PD-L1 inhibitors (e.g., anti-PD-1 and/or anti-PD-L1) suppress the physiologic response to PD-1 and/or PD-L1. The use of immune checkpoint inhibitors that block association/interaction, e.g., binding, of PD-1 and PD-L1 increases the ability of T cells to attack and kill cancer cells and, in turn, decreases the ability of cancer cells to evade the immune system. These inhibitors restore immune function through activation of T-cells and cell-mediated immune responses against cancer/tumor cells.

Inhibition of PD-1 and/or PD-L1 effects can be achieved with monoclonal antibody that target either PD-1 or PD-L1 to block their binding and, in turn, boost the immune response against cancer cells. Drugs that target these checkpoints can be potentially effective for treating cancer cells. Non-limiting examples of known PD-1 inhibitors include Pembrolizumab, Nivolumab and Cemiplimab, which are commercially available under the trade names Keytruda®, Opdivo® and Libtayo®, respectively. These drugs have been used in the treatment of cancer including melanoma of the skin, non-small cell lung cancer, kidney cancer, bladder cancer, head and neck cancers, and Hodgkin lymphoma. Non-limiting examples of known PD-L1 inhibitors include Atezolizumab, Avelumab and Durvalumab, which are commercially available under the trade names Tecentriq®, Bavencio® and Imfinzi®, respectively. These drugs have been used to treat cancer including bladder cancer, non-small cell lung cancer and Merkel cell skin cancer (Merkel cell carcinoma).

There is a concern that known cancer treatment drugs allow the immune system to attack normal organs in the body, in addition to the targeted cancer cells. This concern can lead to serious side effects in patients that are taking the drugs. The serious effects can include serious problems in the lungs, intestines, liver, kidneys, hormone-making glands and other organs.

According to published data, i.e., from the American Cancer Society, it is estimated that 1,665,540 new cancer cases will be diagnosed and 585,720 cancer deaths will occur in 2014, in the United States. Cancer remains the second most common cause of death in the United States and accounts for nearly 1 of every 4 deaths.

Known cancer treatment procedures can be expensive, time-consuming and invasive. Further, these known procedures may not be capable of regressing, reducing or eliminating cancer in certain patients.

Thus, there is a need in the art to develop new cancer therapies and methods of administering the cancer therapies to regress, reduce or eliminate tumor cells in tumor tissue of a patient. It is desirable for the therapies and methods to be effective in a reasonable period of time and further, desirable for the therapies and methods to be as minimally invasive to the patient as reasonably possible. Furthermore, it is advantageous for the therapy and methods to be effective in the presence or absence of subjecting the patient to conventional therapy regimens, such as, radiotherapy and/or chemotherapy. It is an object of the invention to provide drugs and cancer treatments that are effective to target PD-1 or PD-L1 while not causing serious side effects to the patient.

### SUMMARY OF THE INVENTION

The invention resolves the above needs by providing effective therapies and methods for regression, reduction or elimination of tumor cells in tumor tissue of a patient, as well as tumor cells in metastasized tumors. In one aspect, the invention provides a method of introducing intratumorally and/or through vessel a therapeutically effective amount of immature dendritic cells and/or CTLs to the patient, and introducing intratumorally and/or through vessel and/or subcutaneously a therapeutically effective amount of anti PD-1/PD-L1 antibody to the patient.

In certain embodiments, the method can further include collecting monocyte cells and/or the CTLs from a patient, culturing the monocyte cells and/or the CTLs, forming immature dendritic cells from the monocyte cells, and re-introducing the immature dendritic cells and/or the cultured CTLs to the patient. The re-introducing of the cultured CTLs can be conducted prior to or following or coincident with the introducing of the immature dendritic cells.

The monocyte cells can be cultured in a medium including IL-4, GM-CFS, and mixtures thereof to form the immature dendritic cells. The CTLs can be cultured in a medium including IL-2, CD3, and mixtures thereof.

In certain embodiments, the introducing of the anti PD-1/PD-L1 antibody can be coincident with the introducing of the immature dendritic cells and/or CTLs. In other embodiments, the introducing of the anti PD-1/PD-L1 antibody can be immediately following or following a short time thereafter the introducing of the immature dendritic cells and/or CTLs. A short period of time can include seconds or minutes or hours or days. The anti PD-1/PD-L1 antibody may be introduced when there is a sufficient amount of CTLs present in the auto-immune system of the patient.

The introducing of the immature dendritic cells and/or CTLs, and/or the introducing of the anti PD-1/PD-L1 antibody can be in conjunction with an anti-inflammatory agent.

The introducing of the immature dendritic cells and/or CTLs can be in conjunction with an adjuvant. The immature dendritic cells and/or CTLs and adjuvant can be combined to form a composition, and the composition can be introduced intratumorally and/or through vessel into the patient. The adjuvant can be selected from the group consisting of lipid-based, protein-based and polysaccharides-based adjuvants, such as lymphocyte cultured medium, Marignase, Agaricus, OK432, BCG, Lentinan (shiitake), Reishi, Sarunokoshikake, TNF Meshimakobu, Froint's complete or incomplete adjuvant, LPS, fatty acids, TW80, phospholipids, cytokines or a virus, and mixtures thereof. In certain embodiments, the adjuvant can be a leukocyte cultured medium (LCM) adjuvant. The LCM adjuvant can include at least three cytokines selected from the group consisting of eotaxin, FGF, G-CSF, GM-CSF, IFNγ, IP10, IL1β, IL1ra, IL2, IL4, IL5, IL6, IL7, IL8, IL9, IL10, IL12, IL13, IL15, IL17, MCP1, MIP1α, MIP1β, PDGFbb, RANTES, TNFα and VEGF.

In another aspect, the invention provides a method of regressing, reducing or eliminating tumor cells in a patient, which includes obtaining monocyte cells from the patient by isolating the monocyte cells from peripheral blood mononuclear cells, differentiating the monocyte cells to produce immature dendritic cells, combining a first sample of the immature dendritic cells with adjuvant and keyhole limpet to form a first mixture of the immature dendritic cells, introducing intratumorally and/or through vessel the first mixture of the immature dendritic cells into the patient, preparing CTLs from the monocyte-depleted peripheral blood mononuclear cells, introducing intratumorally and/or through vessel a first sample of the CTLs into the patient subsequent to introducing the first mixture of immature dendritic cells, combining a second sample of the immature dendritic cells with adjuvant to form a second mixture of the immature dendritic cells, introducing intratumorally and/or through vessel the second mixture of the immature dendritic cells to the patient, and introducing intratumorally and/or through vessel a second sample of the CTLs to the patient subsequent to introducing the second mixture of the immature dendritic cells Furthermore, the method includes introducing intratumorally and/or through vessel and/or subcutaneously a therapeutically effective amount of anti PD-1/PD-L1 antibody into the patient.

### BRIEF DESCRIPTION OF THE DRAWINGS

A further understanding of the invention can be gained from the following description of the preferred embodiments when read in conjunction with the accompanying drawings in which:

FIG. 1A is a schematic showing effectiveness of anti-PD-1 and/or anti-PD-L1 to suppress/inhibit the interaction and binding of PD-1 and/or PD-L1 wherein view (a) shows inhibited antitumor immunity with PD-1 and PD-L1 and view (b) shows enhanced antitumor immunity with anti-PD-1 antibody and anti-PD-L1 antibody; FIG. 1B shows images of antitumor immunity wherein view (c) shows a dendritic cell and memory T-cell and view (d) shows an autologous cancer cell and CTL, in accordance with certain embodiments of this invention;

FIG. 2 is a plot showing a cancer treatment protocol and the number of tumors for every 3-months check-ups of a female cancer patient, in accordance with certain embodiments of this invention;

FIG. 3A is an image showing conditions of cancer tumors of the female patient before administration of treatment protocol HITV; FIG. 3B is a plurality of images showing before and after conditions of the cancer tumors of the female patient administered the cancer treatment protocol as shown in FIG. 2 wherein views (a) and (b) show before and after IMRT, respectively, views (c) and (d) shown before and after RFA, respectively, views (e) and (f) show before and after DC, respectively, and views (f) and (g) show before and after DC+PD-1, respectively, in accordance with certain embodiments of the invention;

FIG. 4 is a plot showing a cancer treatment protocol and the number of tumors for every 3-months check-ups of a female cancer patient as shown, in accordance with certain embodiments of this invention;

FIG. 5A is a plurality of images in views (a), (b) and (c) showing the female patient's lymphoma tumors on Jan. 1, 2016, and FIG. 5B shows images in view (c), (d) and (e) on Apr. 22, 2016, indicative of the reduction of the lymphoma of the female patient administered of a cancer treatment protocol as shown in FIG. 4, in accordance with certain embodiments of the invention;

FIG. 6A is an image showing a carcinoma of a male patient on Apr. 13, 2016, and FIG. 6B is an image showing reduction of the carcinoma of the male patient on Jun. 15, 2016, as a result of administration of a cancer treatment protocol, in accordance with certain embodiments of the invention;

FIG. 7A is an image showing a carcinoma of a female patient on Feb. 5, 2016, and FIG. 7B is an image showing reduction of the carcinoma of the female patient on May 13, 2016, as a result of administration of a cancer treatment protocol, in accordance with certain embodiments of the invention; and

FIG. 8A is a plot comparison for five assays on tumor cell staining (whole cohort) and FIG. 8B is a plot comparison for five assays on tumor cell staining (non-small cell lung cancer, cytology excluded), for the various cancer protocols shown in Table 7, in accordance with certain embodiments of the invention.

### DETAILED DESCRIPTION OF PREFERRED EMBODIMENTS

The invention includes introducing to a patient immature dendritic cells and/or cytotoxic T lymphocytes (CTLs) and PD-1 and/or PD-L1 inhibitor (anti PD-1/PD-L1 antibody). The immature dendritic cells and/or CTLs are introduced to the patient intratumorally and/or through vessel, and the PD-1 and/or PD-L1 inhibitor is introduced to the patient intratumorally and/or through vessel and/or subcutaneously. The immature dendritic cells may be introduced coincident with introduction of the CTLs. Alternatively, the immature dendritic cells may be introduced prior to (e.g., immediately before or a short time before), or following (e.g., immediately thereafter or a short time thereafter), introduction of the CTLs. Similarly, the CTLs may be introduced coincident with introduction of the immature dendritic cells; alternatively, the CTLs may be introduced prior to (e.g., immediately before or a short time before), or following (e.g., immediately thereafter or a short time thereafter), introduction of the immature dendritic cells. The time between introducing the immature dendritic cells and/or CTLs can vary. In certain embodiments, the time period may range from zero (they are introduced simultaneously) to several seconds or zero to several minutes or zero to several hours or zero to several days or zero to several weeks. Further, the PD-1 and/or PD-L1 inhibitor may be introduced coincident with introduction of the immature dendritic cells and/or the CTLs. Alternatively, the PD-1 and/or PD-L1 inhibitor may be introduced immediately following introduction of the immature dendritic cells and/or the CTLs, or a short time thereafter the introduction of the immature dendritic cells and/or the CTLs, or a longer time thereafter the introduction of the immature dendritic cells and/or the CTLs.

Certain embodiments of the invention include a three phase cancer treatment method or protocol. The three phases are vaccination phase, CTL induction phase, and maintenance phase. The vaccination phase includes a first introduction of immature dendritic cells followed by radiation therapy to the patient, which is followed by a second introduction of immature dendritic cells to the patient. The CTL induction phase includes the introduction of immature dendritic and CTLs to the patient. The maintenance phase includes the introduction of the PD-1 and/or PD-L1 inhibitor to the patient. The maintenance phase may also include the introduction of dendritic cells with the PD-1 and/or PD-L1 inhibitor. The immature dendritic cells and CTLs may be produced by collecting monocyte cells and/or CTLs from a patient; culturing the monocyte cells and/or culturing/activating the CTLs collected from the patient; forming immature dendritic cells from the monocyte cells; and forming cultured CTLs from the natural CTLs. As aforementioned, the immature dendritic cells and CTLs may be introduced intratumorally and/or through vessel, and the PD-1 and/or PD-L1 inhibitor is introduced to the patient intratumorally and/or through vessel and/or subcutaneously.

The administration and combination of the three phases are described herein in additional detail.

The therapies and methods of the invention are effective to induce regression, reduction or elimination of cancerous tumor cells, cancerous tumor tissue, and one or more cancerous tumors including those formed by metastasis. Further, this regression, reduction or elimination can be achieved in the presence or absence of conventional therapy, such as, but not limited to, radiotherapy and chemotherapy. In certain embodiments, therapies and methods of the invention are administered to a patient absent of subjecting the patient to radiotherapy and/or chemotherapy.

As used herein, “patient(s)” include mammal(s), which include human(s).

As used herein, the term “therapeutically effective amount” refers to that amount of CTLs, PD-1 and/or PD-L1 inhibitor, immature dendritic cells, anti-inflammatory agent, adjuvant, TNF/IL-10 inhibitor or combinations thereof, required to bring about a desired effect in a human or other mammal. In all instances, at its most basic level, the desired effect is a regression, reduction or elimination of tumor cells in tumor tissue of the cancer patient when compared to the tumor cells in the tumor tissue of the cancer patient prior to employing the therapies and methods of the invention.

As used herein, the term “subcutaneous” and related terms employing “subcutaneous” or “subcutaneously” refer to therapy that includes the introduction of PD-1 and/or PD-L1 inhibitor beneath the skin of a patient, e.g., hypodermic.

As used herein, the term “through vessel” and related terms employing “through vessel” refer to therapy that includes the introduction of immature dendritic cells and/or CTLs and/or PD-1 and/or PD-L1 inhibitor into a channel, such as, a vein or artery, in a patient's body for carrying fluid.

As used herein, the term “intratumoral therapy” and related terms employing “intratumoral” or “intratumorally” refer to therapy that includes the introduction (e.g., injection) of immature dendritic cells and/or CTLs and/or PD-1 and/or PD-L1 inhibitor directly into the tumor tissue of a patient.

The PD-1 and/or PD-L1 inhibitor can be in various forms known in the art. The PD-1 and/or PD-L1 inhibitor can be incorporated into a delivery mechanism, such as a carrier or medium, to facilitate introduction into the patient. In certain embodiments, the PD-1 and/or PD-L1 inhibitor can be included or contained in a liquid for ease of introduction, e.g., injection.

In general, PD-1 and PD-L1 are referred to as checkpoint proteins because they keep the immune system in check. The PD-1 is found on T cells, e.g., CTLs, and PD-L1 is found on cells including cancer cells. As aforementioned, the association or interaction of PD-1 and PD-L1, such as when PD-1 binds to PD-L1, results in PD-1 acting as an “off switch” or “brake” to prevent or block the CTLs from attacking other cells in the body. Since large amounts of PD-L1 can be found on some cancer cells, the binding of PD-1 with PD-L1 contributes to the ability of cancer cells to evade immune system attack.

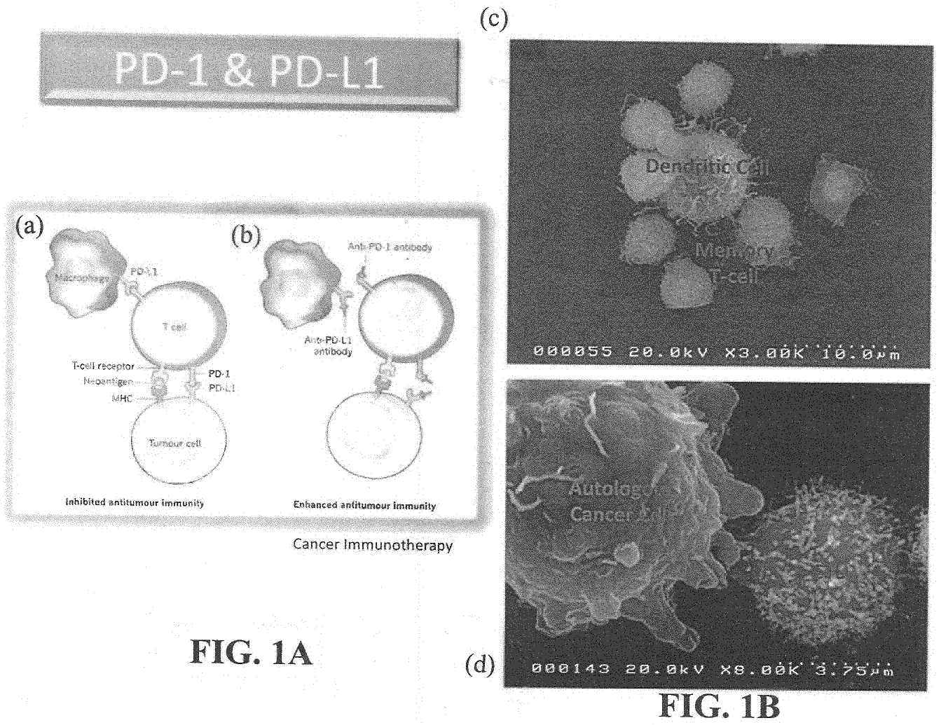

It has been found that PD-1 and/or PD-L1 inhibitors, e.g., anti-PD-1 and/or anti-PD-L1, can be effective to suppress/inhibit the interaction and binding of PD-1 and PD-L1, and some monoclonal antibodies are effective PD-1 and/or PD-L1 inhibitors. These monoclonal antibodies that target either PD-1 or PD-L1 can block their binding and therefore, boost the immune response against cancer cells. As shown in FIG. 1A, view (a), antitumor immunity is inhibited and as shown in FIG. 1A, view (b), antitumor immunity is enhanced. Thus, in accordance with the invention, anti PD-1/PD-L1 antibody is combined with CTLs to inhibit binding and, in turn, preclude evasion of immune attack. In certain embodiments, the combination of anti PD-1/PD-L1 antibody and CTLs is coincident, such as being co-introduced or co-injected in the patient.

In the therapies and methods of the invention, introduction of anti PD-1/PD-L1 antibody intratumorally and/or through vessel and/or subcutaneously is effective to suppress the binding of PD-1 and PD-L1. Suppression of this binding, the CTLs, e.g., induced by the introduction of the immature dendritic cells and/or CTLs, are not blocked from attacking and/or killing cancer cells in the body, and may be effective to destroy tumor cells both locally and systemically in the patient. The result of this immunological function can vary and may depend on the amount of tumor cells, and CTLs in the patient's body. In certain embodiments, these factors are used to determine a therapeutically effective amount of anti PD-1/PD-L1 antibody to be introduced or administered to the patient in order to achieve a favorable outcome, e.g., to maximize the regression, reduction or elimination of tumor cells (local and metastasized) in the patient. It is contemplated that an appropriate balance of the amount of tumor cells and PD-1 and/or PD-L1 present in the patient's body, the amount of CTLs induced by the introduction intratumorally and/or through vessel of immature dendritic cells and/or CTLs, and the amount of anti PD-1/PD-L1 antibody introduced intratumorally and/or through vessel and/or subcutaneously in the patient's body results in maximizing the effect of the therapy and the regression of tumors.

It is found that the therapies and methods of the invention which control, e.g., suppress, the binding of the PD-1 and PD-L1 are effective to treat tumors. In certain embodiments, the therapies and methods of the invention are particularly effective to treat multiple metastasized tumors.

Further, without intending to be bound by any particular theory, it is believed that CTLs, which are naturally induced as part of the autoimmune response above-described, may not be potent against tumors especially when the tumors are at an advanced stage or aggressively growing. The reasons may be that these CTLs are not induced in an adequate quality and/or in an adequate quantity and/or in a timely manner to defend the patient's body from the tumor invasion. Since CTLs are produced by the patient, in certain embodiments, CTLs can be collected from the patient, cultured, and then introduced, e.g., returned, to the same patient's body. The culture medium can vary and may be selected from those known in the art. Non-limiting examples include, but are not limited to, IL-2, CD3, and mixtures thereof.

In certain embodiments, the invention includes collecting monocyte cells from the patient. The monocyte cells are cultured to form immature dendritic cells which are then introduced, e.g., returned, to the same patient's body. The culture medium can vary and may be selected from those known in the art. Non-limiting examples include, but are not limited to, IL-4, GM-CFS, and mixtures thereof. In certain embodiments, CTLs are collected from the patient following collection, culture and return of the immature dendritic cells into the patient. Subsequent to their collection, the CTLs are cultured in a suitable medium and then the cultured CTLs are introduced, e.g., returned, to the same patient's body. The immature dendritic cells and/or CTLs are introduced intratumorally and/or through vessel into the patient. In alternative embodiments, only immature dendritic cells are introduced or only CTLs are introduced or a combination of immature dendritic cells and CTLs are introduced into the patient.

The anti PD-1/PD-L1 antibody can be introduced intratumorally and/or thorough vessel and/or subcutaneously into the patient in a therapeutically effective amount prior to, coincident with, or following introducing the cultured CTLs and/or immature dendritic cells. The amount or dose of anti PD-1/PD-L1 antibody administered can vary and may depend on the type and progression of the cancer, as well as the mechanism for administration/introduction. In certain embodiments, about 10 mg (e.g., 10 mg/ml) or about 20 mg (e.g., 20 mg/ml) of the anti PD-1/PD-L1 is injected intratumorally or an amount within the range of about 10 mg (e.g., 10 mg/ml) to about 20 mg (e.g., 20 mg/ml) is injected intratumorally. For intravenous administration, e.g., DIV/IV infusions, about 50 mg or about 100 mg of the anti PD-1/PD-L1 is introduced or an amount within the range of about 50 mg to about 100 mg is introduced. The anti PD-1/PD-L1 may be a single administration or dose, e.g., injection or infusion, or may include multiple administrations or doses within a treatment protocol.

In accordance with the invention, an adequate quantity and quality of CTLs are provided in the patient's body and, in particular, at the tumor site(s), to regress, reduce or eliminate tumor cells. Further, the anti PD-1/PD-L1 antibody is introduced to inhibit binding of PD-1 and PD-L1, and in turn inhibit “turning off” or blocking the CTLs from attacking cancer cells in the body. Further, the invention provides for collecting the natural CTLs from the patient, culturing or activating these collected cells, and re-introducing them, e.g., and anti PD-1/PD-L1 antibody, into the same patient in a quantity and quality which is sufficient (e.g., a therapeutically effective amount) to regress, reduce or eliminate tumor cells.

In certain embodiments, the CTLs include CD8+NK T cell population.

In certain embodiments, the invention is a human-initiated therapeutic vaccine with immature dendritic cells and/or CTLs in combination with anti-PD-1 and/or anti-PD-L1 antibody.

In certain embodiments, the immature dendritic cells and/or CTLs are introduced in conjunction with an adjuvant. The adjuvant can be introduced intratumorally and/or through vessel into the patient prior to, coincident with or following intratumoral and/or through vessel introduction of the immature dendritic cells and/or CTLs. In certain embodiments, the immature dendritic cells and/or CTLs and the adjuvant can be combined to form a composition that can be introduced intratumorally and/or through vessel to the patient. Further, the immature dendritic cells and/or CTLs, and optionally adjuvant, are introduced intratumorally and/or through vessel prior to, coincident with or following introducing intratumorally and/or through vessel and/or subcutaneously the anti PD-1/PD-L1 antibody to the patient.

Suitable adjuvants for use in the invention can include, without limitation, lipid-based, protein-based and polysaccharides-based adjuvants, such as lymphocyte cultured medium, Marignase, Agaricus, OK432, BCG, Lentinan (shiitake), Reishi, Sarunokoshikake, TNF Meshimakobu, Froint's complete or incomplete adjuvant, LPS, fatty acids, TW80, phospholipids, cytokines or a virus. In certain embodiments, the adjuvant can be a leukocyte cultured medium (LCM) adjuvant. The LCM adjuvant can include at least three cytokines selected from the group consisting of eotaxin, FGF, G-CSF, GM-CSF, IFNγ, IP10, IL1β, IL1ra, IL2, IL4, IL5, IL6, IL7, IL8, IL9, IL10, IL12, IL13, L15, IL17, MCP1, MIP1α, MIP1β, PDGFbb, RANTES, TNFα and VEGF.

In certain embodiments, the immature dendritic cells and/or CTLs can be introduced to the patient in conjunction with an anti-inflammatory agent. Suitable anti-inflammatory agents can include those that are known in the art. The anti-inflammatory agent can be introduced prior to, coincident with or following introduction of the immature dendritic cells and/or CTLs. It is typical for introduction of the immature dendritic cells, CTLs and anti-inflammatory agent to be simultaneous or substantially simultaneous, or for the elapsed time between introducing the immature dendritic cells and/or CTL and the anti-inflammatory agent to be relatively short in duration.

The invention can optionally include a precursor treatment. That is, prior to introducing the dendritic cells and/or CTLs, the patient may be administered a treatment selected from chemotherapy, radiotherapy, antibody therapy, and combinations thereof. These regimens are well known in the art. Further, optionally, it is contemplated that the use of these regimens may be employed at various other times throughout the method of the invention. However, it is further understood, that these regimens are not necessary. The therapy and methods of the invention are effective to regress, reduce or eliminate tumor tissue in a patient in the absence of chemotherapy, radiotherapy, antibody therapy, and combinations thereof.

The method of administering the therapy according to certain embodiments of the invention includes the following steps.
Step 1: Collecting monocyte cells and/or CTLs from a patient. Various conventional techniques known in the art can be employed for their collection.
Step 2: Culturing the monocyte cells and/or culturing/activating the CTLs collected from the patient. The monocyte cells form immature dendritic cells. Various conventional techniques known in the art can be employed to culture the monocyte cells and/or to culture/activate the CTLs, and various culture mediums known in the art can be used, such as those disclosed herein.
Step 3: Introducing intratumorally and/or through vessel, such as but not limited to injecting, a therapeutically effective amount of the immature dendritic cells and/or CTLs into the patient. The immature dendritic cells and/or CTLs can be mixed or combined with an adjuvant and the mixture or combination can be introduced intratumorally and/or through vessel into the patient. The adjuvant can be selected from those known in the art and disclosed herein. Further, optionally, an anti-inflammatory agent may be present with the immature dendritic cells and/or CTLs and adjuvant as disclosed herein.
Step 4: Introducing intratumorally and/or through vessel and/or subcutaneously, such as, but not limited to, injecting, a therapeutically effective amount of anti PD-1/PD-L1 antibody to the patient. In certain embodiments, from 10 mg to 20 mg of the anti PD-1/PD-L1 antibody is introduced intratumorally and/or through vessel and/or subcutaneously to the patient. This amount may constitute a single dose. Treatment protocols in accordance with the invention, can include the introduction of multiple doses of anti PD-1/PD-L1 antibody wherein each dose is from 10 mg to 20 mg. Further, in certain embodiments, a combination of dendritic cells and anti PD-1/PD-L1 antibody may be introduced.

The time allowed to lapse between and within the above-identified steps can vary. For example, the time allowed to lapse between Steps 3 and 4 can vary. In certain embodiments, Step 3 can be performed coincident with Step 4 and in other alternate embodiments, Step 4 can be performed following Step 3, such as a couple of seconds, hours, days, or weeks after Step 3.

The time allowed to lapse within Step 3, i.e., between the introduction of the immature dendritic cells and CTLs, can also vary. In certain embodiments, their introduction is simultaneous or substantially simultaneous. In other embodiments, there is a pre-determined or desired time period which is allowed to lapse between the introduction of each. It is typical for this period of time to be relatively short in duration.

Further, as previously indicated, the CTLs can be collected from the patient coincident with collection of the monocyte cells or at a time thereafter. In certain embodiments, the monocyte cells are collected and cultured to form immature dendritic cells and the dendritic cells are introduced into the patient prior to collection of CTLs from the patient.

Furthermore, as previously indicated, conventional therapy, such as, radiation or chemotherapy, may be conducted at any time during Steps 1 through 4.

In certain embodiments, the invention can include the treatment steps of: introducing intratumorally and/or through vessel immature dendritic cells in a therapeutically effective amount into the tumor tissue of the patient; collecting from the patient CTLs induced by the introduction of the immature dendritic cells; re-introducing intratumorally and/or through vessel the collected CTLs into the tumor tissue of the same patient; and introducing intratumorally and/or through vessel and/or subcutaneously anti PD-1/PD-L1 antibody to the patient. The introduction of immature dendritic cells may be a prerequisite to the collection and/or introduction of the CTLs cells. In certain embodiments, the introduction of the immature dendritic cells is a prerequisite to introduction of the anti PD-1/PD-L1 antibody. Further, the introduction of the immature dendritic cells may be combined, e.g., simultaneous, with introduction of the anti PD-1/PD-L1 antibody.

In certain embodiments, the inducement of CTLs by the immature dendritic cells is sufficient (e.g., a therapeutically effective amount) such that the natural CTLs are not removed from the patient and not re-introduced into the same patient. Thus, the anti PD-1/PD-L1 antibody can be introduced with the immature dendritic cells (e.g., absent of re-introducing CTLs).

Without intending to be bound by any particular theory, it is believed that immature dendritic cells which are formed by culturing monocyte cells collected from a patient and CTLs which are produced by a patient and collected from the patient, provide for an enhanced desired effect when injected into the same patient as compared to immature dendritic cells and CTLs produced and obtained by other means. It appears that immature dendritic cells which are formed from the patient's own monocyte cells which have been collected and CTLs which have been collected, cultured and re-introduced intratumorally and/or through vessel, provide improved coupling or interaction with other cells in the body of the patient.

In certain embodiments, the invention provides regression, reduction or elimination of tumor cells in tumor tissue which can be visually detected by MRI and/or CT and/or Echo scan.

Further, in certain embodiments of the invention, a combination of immunotherapy and local radiation is administered to a cancer patient. Without intending to be bound by any particular theory, it is believed that this combination of treatments provides for a therapeutic protocol to stimulate a systemic adaptive immune response against malignant cells. In accordance with this protocol, monocyte cells are obtained from the patient for differentiation into immature dendritic cells. The monocyte cells are isolated from peripheral blood mononuclear cells (PBMCs). The monocyte cell-depleted, T-cell enriched fraction of the PBMCs is then used to prepare activated T-cells, e.g., CTLs. The immature dendritic cells are combined with LCM, a multi-cytokine-based adjuvant, and keyhole limpet hemocycanin (KLH). This mixture is injected into the patient. Subsequently, such as but not limited to, on the following day, activated T-cells, e.g., CTLs, are infused. After a period of time, such as, but not limited to, about seven days, local radiation is administered to the patient. After a period of time, such as, but not limited to, about another seven days, a second sample of immature dendritic cells and activated T-cells, e.g., CTLs, is administered to the patient. For example, immature dendritic cells suspended in LCM are injected and then, e.g., on the following day, activated T-cells, e.g., CTLs, are infused. It has been found that this protocol results in an increase in CD8⁺ CD56⁺ cells. Without intending to be bound by any particular theory, it is believed that these cells are capable of killing cancer cells.

Moreover, in certain embodiments, the cancer treatment in accordance with the invention includes the introduction of a therapeutically effective amount of TNF inhibitor (anti TNF), such as TNF antibody, and/or IL-10 inhibitor (anti IL-10), such as IL-10 antibody to the patient. The TNF/IL-10 inhibitor may be administered with the dendritic cells and/or CTLs. For instance, the TNF/IL-10 inhibitor is introduced coincident with one or both of dendritic cells and CTLs; alternatively, the TNF/IL-10 inhibitor is introduced prior to or following introduction of one or both of dendritic cells and CTLs. The TNF/IL-10 inhibitor may be administered intratumorally and/or through vessel and/or subcutaneously to the patient. It is contemplated that the intratumoral injection of immature dendritic cells results in inducement of CTLs as part of an immunoresponse. This immunoresponse may promote a TNF response, which may in turn induce inflammation. Thus, the TNF/IL-10 inhibitor introduced with CTLs, e.g., co-introduced or co-injected, may inhibit or reduce such inflammation.

Accordingly, TNF antibody (anti TNF) is effective to suppress the systemic inflammation caused by TNF and by suppressing the inflammation, the CTLs are not limited in their immunological function. The TNF antibody may be introduced when there is a sufficient amount of CTLs presenting in the autoimmune system of the patient such that the immune response of CTLs is supported by suppressing the activity of the TNF. The TNF antibody can be administered in various forms, such as, incorporated in a delivery mechanism, e.g., liquid carrier/medium, to facilitate introduction/injection to the patient.

The invention includes one or more of the following features:
Anti PD-1/PD-L1 antibody induces tolerance break between cancer cells and CTLs;
A combination of dendritic cells and Nivolumab has been shown to control growth of new tumors through IV/IA infusions (12) or intra-tumoral injections (43); and
A higher regulation rate has been shown by intra-tumoral injection (60.5%) for CR/PR than IV/IA infusion (16.7%) for PR.

### EXAMPLES

As shown by the clinical data in Table 1, multiple cancer patients having a variety of cancer diagnoses (e.g., liver, esophageal, breast, lung, colon, ovarian, rectal, cervical, uterine, lymphoma, pancreatic, and bone) were administered a treatment protocol at the various sites, e.g., organs and tissues, in the body corresponding to the particular diagnosis, in accordance with certain embodiments of the invention. The patients experienced partial response or regression (PR) or complete response or regression (CR) of cancer tumors, or the results showed progressive disease (PD) or stable disease (SD). Table 1 shows that the treatment protocol for most of the patients included administration of dendritic cells and PD-1 inhibitor, which was an anti-PD-1 antibody known generically as Nivolumab and commercially available under the tradename Opdivo®.

Table 2 shows the efficacy of Nivolumab for Stage III lung cancer, Stage IV pancreatic cancer, malignant lymphoma and colon cancer, as well as for various recurrent cancers. In addition, Table 2 demonstrates the favorable results of the treatment protocol including intratumoral administration of Nivolumab. The 43 intratumoral injections resulted in the following outcome: 14 complete responses or regression (CR), 12 partial responses or regression (PR), 1 stable disease (SD) and 16 progressive diseases (PD). Thus, 26 out of the 43 injections, i.e., 60.5%, provided CR or PR. As shown in Table 2, intratumoral injection resulted in a higher regulation rate for CR/PR than the IV/IA infusions. There were 2 partial responses or regression (PR) based on 12 infusions, i.e., 16.7%.

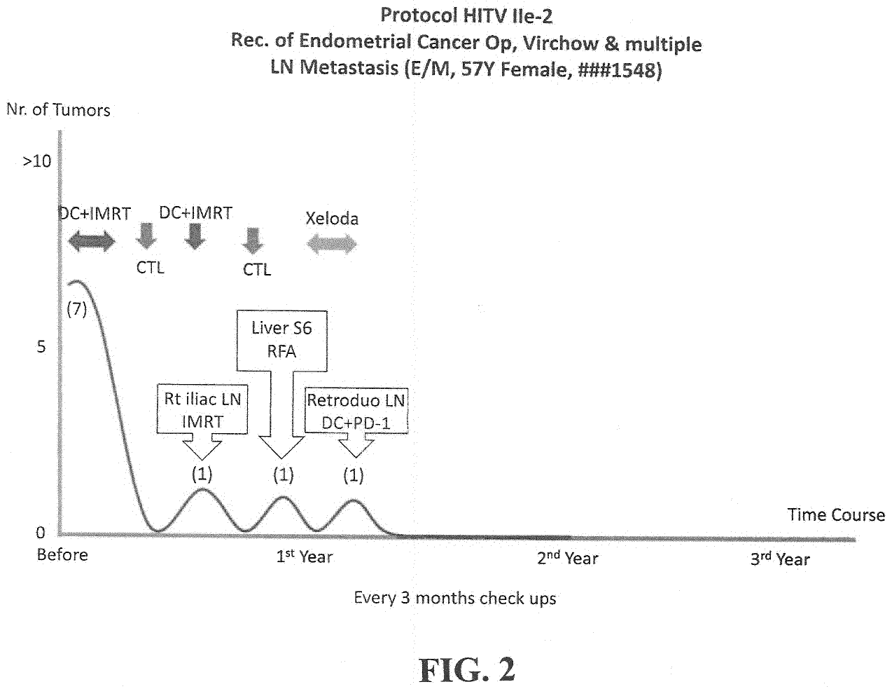

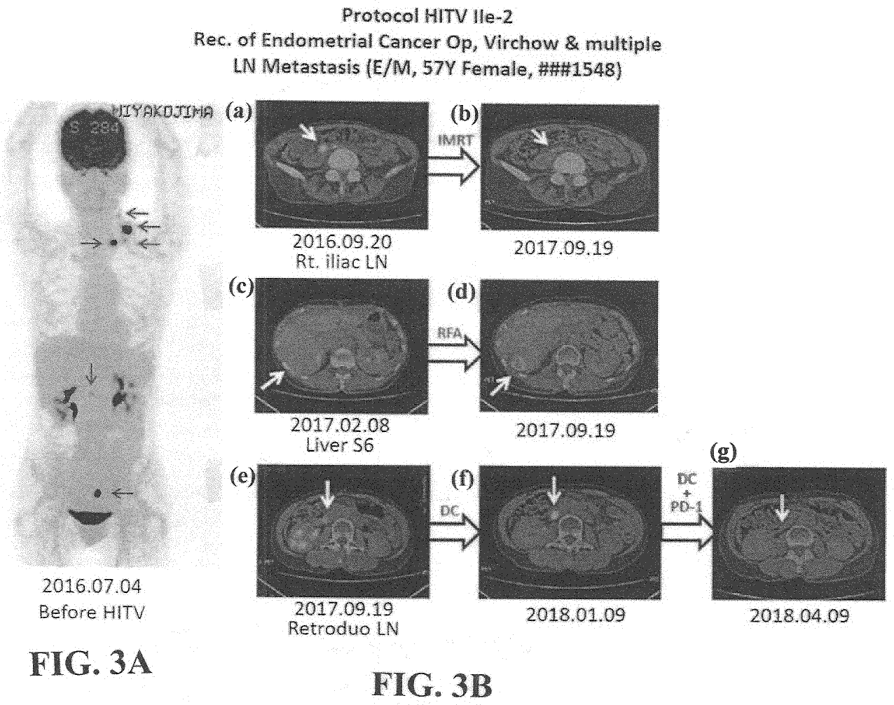

Table 3 shows a treatment protocol in accordance with certain embodiments of the invention, which was administered to a female patient (Patient ID: 1548EM) diagnosed with endometrial cancer and metastasis. Chemotherapy was administered to the patient, followed by apheresis to collect monocyte cells and CTLs from the patient. The monocyte cells and CTLs were cultured. Immature dendritic cells produced from the monocyte cells were introduced to the patient, followed by introduction of the cultured CTLs and subsequently, the introduction of dendritic cells and a PD-1 inhibitor. As shown in Table 3, on Jan. 16, 2018, dendritic cells and Nivolumab (anti-PD-1 antibody) were administered to the patient. As shown in FIG. 2 and FIGS. 3A and 3B (views (a) through (g)), administration of this protocol according to certain embodiments of the invention resulted in a reduction and subsequent elimination of a number of tumors. In FIGS. 3A and 3B (views (a) through (g)), the arrows within the images point to the location of the tumors.

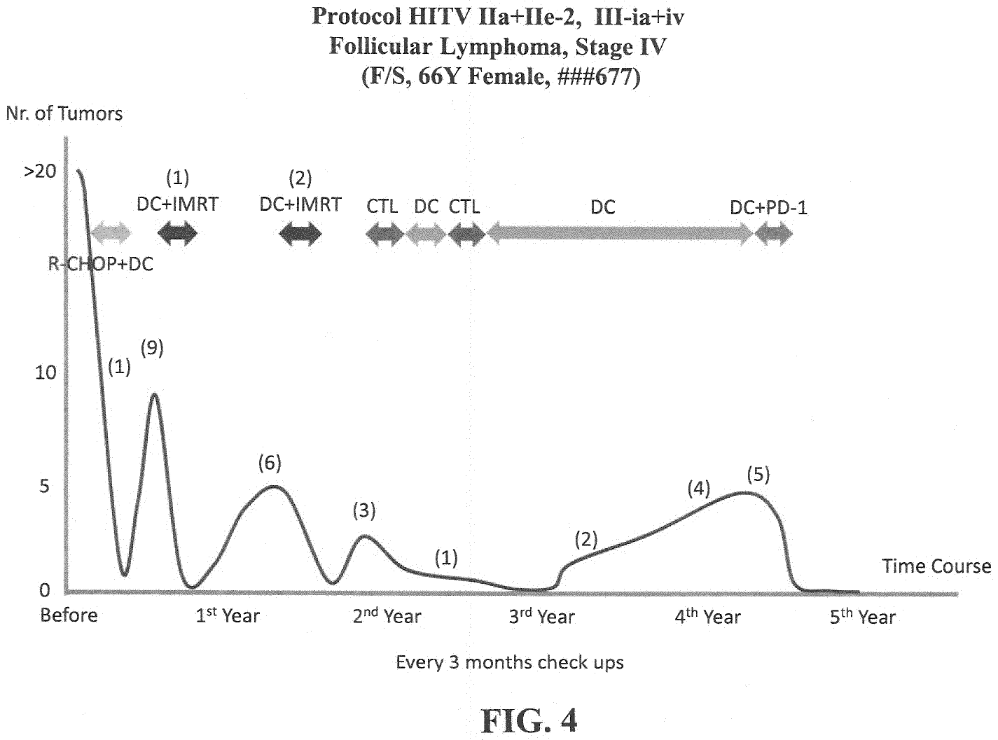

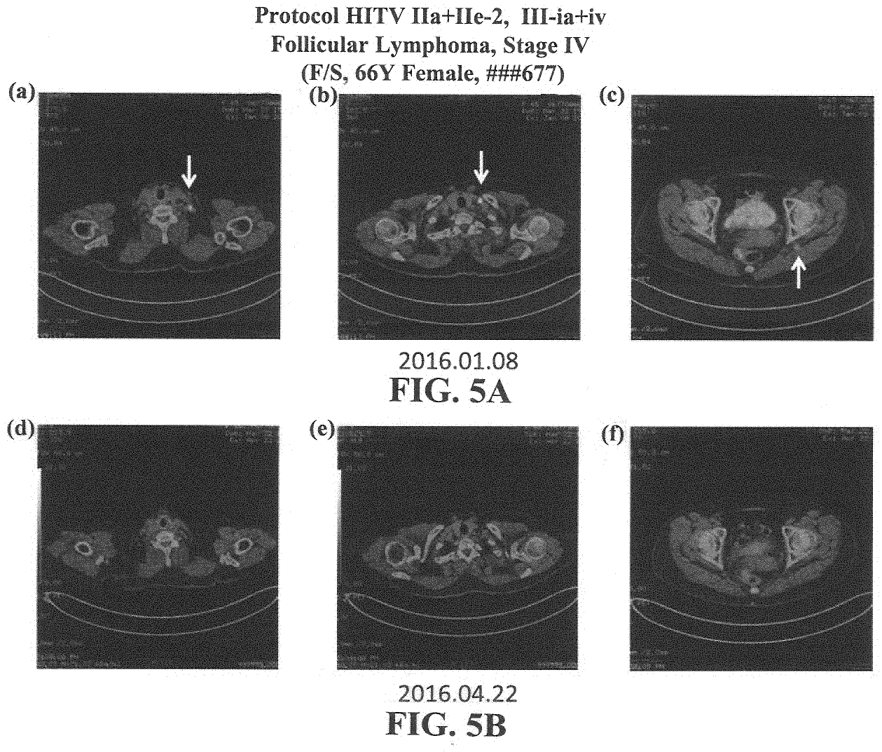

Table 4 shows a treatment protocol in accordance with certain embodiments of the invention, which was administered to a female patient (Patient ID: 0677FS) diagnosed with malignant lymphoma. Chemotherapy was administered to the patient, and apheresis was performed on the patient to collect monocyte cells and CTLs. The monocyte cells and CTLs were cultured. The immature dendritic cells produced from the monocyte cells were introduced to the patient, followed by introduction of the cultured CTLs and subsequently, the introduction of a PD-1 inhibitor. As shown in Table 4, on Feb. 9, 2016, Nivolumab (anti-PD-1 antibody) was administered to the patient. As shown in FIG. 4 and FIGS. 5A (views (a) through (c)) and 5B (views (d) through (f)), administration of this protocol according to certain embodiments of the invention resulted in a reduction and subsequent elimination of a number of tumors. In FIG. 5A (views (a) through (c)), the arrows within the images point to the location of the tumors.

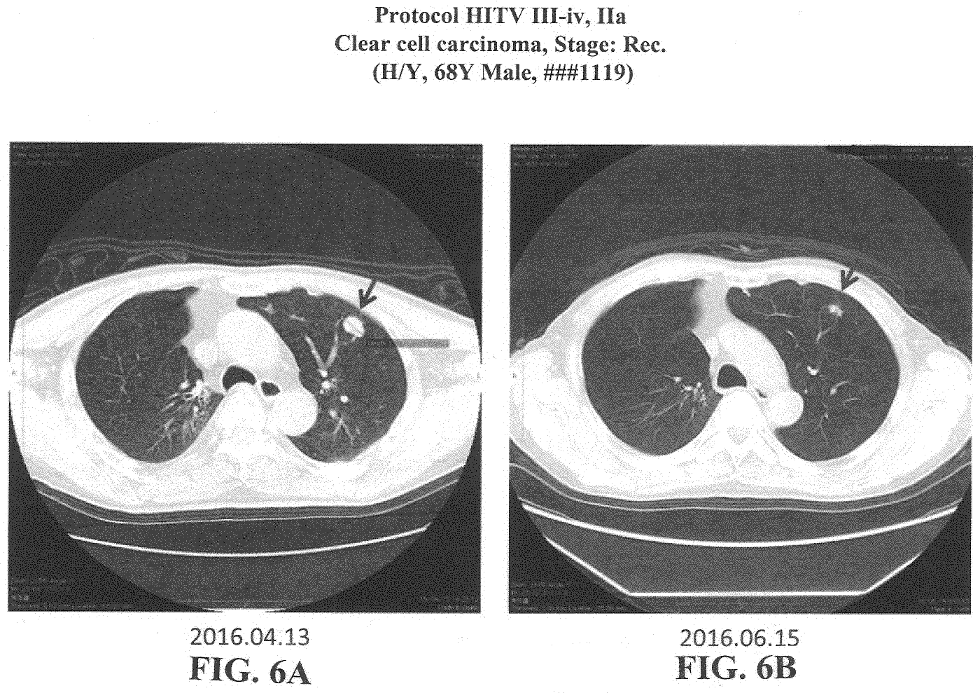

Table 5 shows a treatment protocol in accordance with certain embodiments of the invention, which was administered to a male patient (Patient ID: 1119HY) diagnosed with recurrence of renal cancer with developing multiple lung metastasis. Apheresis was performed on the patient to collect monocyte cells from the patient. The monocyte cells were cultured. The immature dendritic cells produced from the monocyte cells and PD-1 inhibitor were administered on Apr. 13, 2016, Jul. 12, 2016, Oct. 19, 2016, Aug. 22, 2018, and Oct. 22, 2018, as shown in Table 5. On Jan. 21, 2019 and Feb. 18, 2019, dendritic cells and PD-L1 inhibitor (Durvalumab) were administered to the patient. As shown in FIGS. 6A and 6B, administration of this protocol according to certain embodiments of the invention resulted in a significant reduction of clear cell carcinoma. In FIGS. 6A and 6B, the arrows within the images point to the location of the tumors.

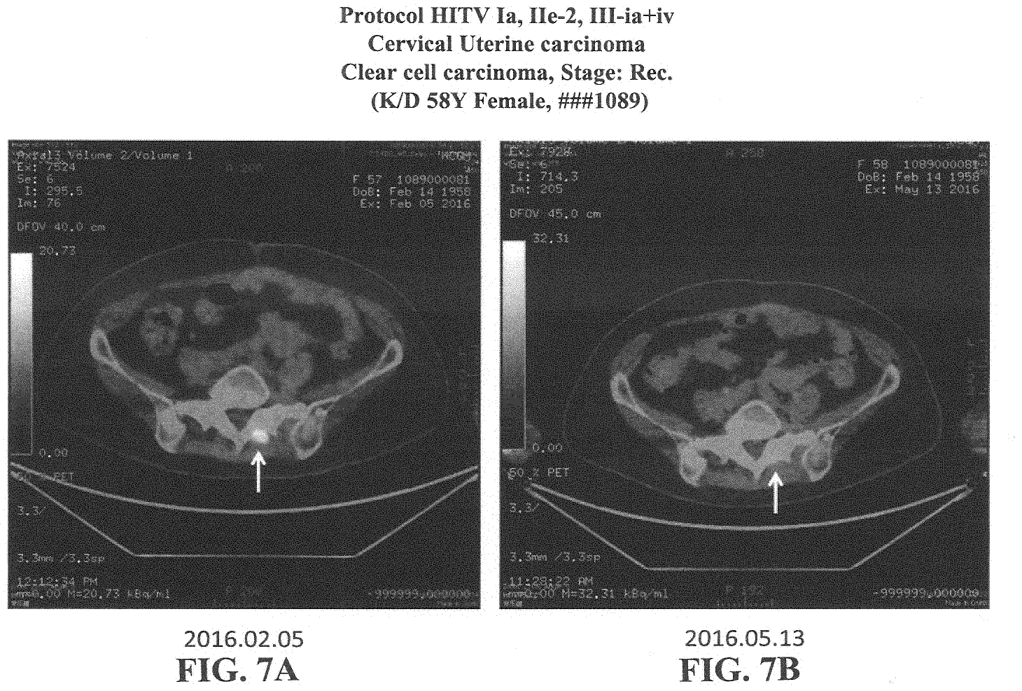

Table 6 shows a treatment protocol in accordance with certain embodiments of the invention, which was administered to a female patient (Patient ID: 1089KD) diagnosed with recurrence of cervical uterine carcinoma. Chemotherapy was administered to the patient, followed by apheresis to collect monocyte cells and CTLs from the patient. The monocyte cells and CTLs were cultured. The immature dendritic cells produced from the monocyte cells were introduced to the patient, followed by introduction of the cultured CTLs and subsequently, the introduction of dendritic cells and a PD-1 inhibitor. Table 6 shows dendritic cells and PD-1 inhibitor were administered on Feb. 18, 2016. As shown in FIGS. 7A and 7B, administration of this protocol according to certain embodiments of the invention resulted in significant regression of the clear cell carcinoma. In FIGS. 7A and 7B, the arrows within the images point to the location of the tumors.

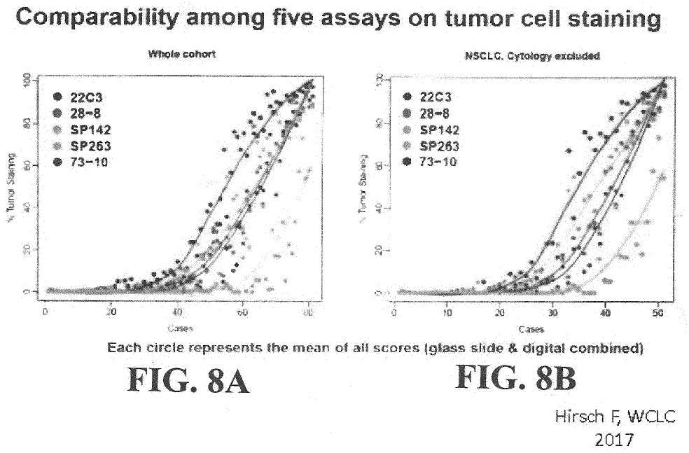

Table 7 shows immune checkpoint inhibitors and corresponding PD-L1 assays. The drugs identified are commercially available under the corresponding trade name and from the associated manufacturer/producer. Nivolumab (Opdivo) and Pembrolizumab (Keytruda) target PD-1, whereas Atezolizumab (Tecentriq), Durvalumab (Imfinzi) and Avelumab (Bavencio) target PD-L1. FIGS. 8A and 8B show two plots to compare these five assays on tumor cell staining.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

TABLE 1
Nivolumab (antiPD-1) Clinical Data

| Patient ID | Diagnosis | Treated Sites | DC | anti-PD-1 | Ad. Date | Method | Ch-T | Ev. Time | Result |
|---|---|---|---|---|---|---|---|---|---|
| #1177SC | Rec. of Mailgnant | Pleura | | 100 mg | 2016 Jan. 21 | DV | No | 3M | PR |
| | Thymoma Op. | | | | | | | | |
| #1404NS | Rec. of esophageal Ca. Op | Multiple LN Meta. | | 100 mg | 2016 Jan. 20 | DIV | Yes | 1M | PD |
| | | Multiple LN Meta. | | 50 mg | 2016 Apr. 20 | DIV | No | 2M | PD |
| | | LN × 4 | 4U | 10 mg × 4 | 2016 Jun. 06 | IT | No | 5M | PD |
| | | LN × 4 | 4U | 10 mg × 4 | 2016 Jul. 05 | IT | No | | PD |
| #918TW | Rec. of Adenocystic NPC Op. | Lung & Pleura | | 100 mg | 2006 Jan. 21 | DIV | Yes | 4 H | PD |
| | Lung & Pleural Meta. | | | | | | | | |
| #1316TH | Rec. of Gastric Ca. Op. | Liver, Bil-adrenal | 2U | 100 mg | 2016 Jan. 20 | DIV | No | 2M | PD |
| | | LN Meta. Ascitis | | | | | | | |
| #1212RK | Rec of right breast Ca Op. | Left Pleura | | 100 mg | 2016 Jan. 26 | DIV | Yes | 1M | PR |
| | | | 1U | 100 mg | 2016 Mar. 22 | DIV | Yes | 1M | SD |
| | | | 1U | 50 mg | 2016 Apr. 19 | DIV | Yes | 1M | SD |
| | | Botallo LN, Pleura | 2U | 10 mg × 2 | 2016 May 19 | IT | Yes | 1M | PR |
| | | Th5, Pleura | 2U | 10 mg × 2 | 2016 Jun. 15 | IT | Yes | 1.5M | PR |
| | | Pleura × 2 | 2U | 10 mg × 2 | 2016 Aug. 22 | IT | No | 1M | PD |
| #1340KD | Pancreatic Ca. | | 3U | 100 mg | 2016 Jan. 15 | DIV | Yes | 1M | PD |
| | Liver, LN metastasis | | | | | | | | |
| #677FS | Malignant Lymphoma | Lt. SCLN × 2 | 2U | 10 mg × 2 | 2016 Feb. 09 | IT | No | 2M | CR |
| | (St. IV) | Lt. Giuteal LN | 1U | 10 mg | 2016 Feb. 09 | IT | No | 2M | CR |
| #1122MW | Rt. Lung Ca. | Rt. S6 Primary | 1U | 10 mg | 2016 Feb. 24 | IT | No | 0.5M | PR |
| | | Rt. S6 Primary | 1U | 10 mg | 2016 Mar. 30 | IT | No | 3M | CR |
| | | Rt. Pleural Effusion | 1U | 10 mg | 2016 Jun. 21 | IP | No | 2M | CR |
| #1326AA | Rec. of Breast Ca. Op. | Rt. SCLN | 1U | 10 mg | 2016 Jan. 20 | IT | No | 2M | SD |
| #1000MN | Rec. of Malignant Thymoma | Lt. Upper Med. LN | 1U | 10 mg | 2016 Mar. 08 | IT | No | 1M | PR |
| | | Paratracheal LN | 1U | 10mg | 2016 Mar. 08 | IT | No | 2M | CR |
| | | Lt. SCLN × 3 | 3U | 10 mg × 3 | 2016 Apr. 05 | IT | No | 1M | PR |
| #1210AO | Colon Ca. | Ovarium | 2U | 20 mg | 2016 Feb. 24 | IT | Yes | 3M | PR |
| | Lung, Liver & Ovarial Meta. | | | 50 mg | 2016 Apr. 20 | DIV | Yes | 1.5M | PD |
| | | Botallo LN | 1U | 10 mg | 2016 Jun. 27 | IT | Yes | 2M | PD |
| | | Rt. Lung S3 | 1U | 10 mg | 2016 Jun. 27 | IT | Yes | 2M | PD |
| | | Liver S3 & S8 | 2U | 10 mg × 2 | 2016 Jul. 25 | IT | Yes | 1M | PD |
| #910KK | Rec. of Rectal Ca. Op. | Rt. Ilian LN | 1U | 100 mg | 2016 Jan. 21 | DIV | Yes | 6M | PD |
| | Peritoneal Dissemination | Rt. Iliac LN | 1U | 40 mg | 2016 May 12 | 1A | Yes | 1.5M | PD |
| | Sacrum & Dauglas Meta. | Rt, Iliac LN × 2 | 2U | 10 mg × 2 | 2016 Aug. 30 | IT | Yes | | PD |
| #1089AD | Rec. of Cervical Uteririe | Sacral Soft Tissue | 1U | 10 mg | 2016 Feb. 18 | IT | No | 3M | CR |
| | Ca. Op. | | | | | | | | |
| | Lt. Pelvic LN & Sacral | | | | | | | | |
| | bone Met. | | | | | | | | |
| #433YI | Rec. of Rt. Breast Ca. Op. | Th11 | 1U | 10 mg | 2016 Mar. 29 | IT | No | 2M | CR |
| | Multi. Bone Meta. | Parasternal LN | 1U | 10 mg | 2016 Mar. 29 | IT | No | 2M | CR |
| #1119HY | Rec. of Lt. Renal Ca. | Lt. Lung S3 | 2U | 20 mg | 2016 Apr. 13 | IT | No | 2M | PR |
| | Mult. Lung Met. | | | | | | | | |
| #884SM | Rec. of Lt. Breast Ca. Op. | PALN | 1U | 10 mg | 2016 Apr. 26 | IT | No | 2M | PR~CR |
| | Mult. LN Meta> | PALN | 1U | 10 mg | 2016 Jun. 02 | IT | No | 1M | CR |
| #677FS | Malignant Lymphoma | Lt. Glutal LN | 1U | 10 mg | 2016 Feb. 09 | IT | No | 3M | CR |
| | (St. IV) | Lt. SCLN | 1U | 10 mg | 2016 Feb. 09 | IT | No | 3M | CR |
| | | Lt. ICLN | 1U | 10 mg | 2016 Feb. 09 | IT | No | 3M | CR |

TABLE 2
Diagnosis and Efficacy of Nivolumab for 17 Cases

| Stage III | | | Stage IV | | Recurrent | | |
|---|---|---|---|---|---|---|---|
| Lung Ca | 1 | | Pancreatic Ca | 1 | Malig. Thymoma | 2 | |
| | | | Mali. Lymphoma | 2 | Esophageal Ca | 1 | |
| | | | Colon Ca | 1 | NPC | 1 | |
| | | | | | Gastric Ca | 1 | |
| | | | | | Breast Ca | 4 | |
| | | | | | Rectal Ca | 1 | |
| | | | | | Uterus Ca | 1 | |
| | | | | | Renal Ca | 1 | |
| DIV or 1A (12 Infusions) | | | | IT: Intra-tumora (43 Injections) | | | |
| CR | PR | SD | PD | CR | PR | SD | PD |
| 0 | 2 | 2 | 8 | 14 | 12 | 1 | 16 |

| | |
|---|---|
| 16.7% | 60.5% |

TABLE 3
Patient ID: 1548EM
Gender: Female
DOB: 1960/02/06
Clinical Diagnosis: Rec. of endometrial cancer,Virchow & multiple LN metastasis
Pathology: adenocarcinoma
Stage: Rec.
Present History:

| | |
|---|---|
| 2015.11 | Endometrial cancer diagnosed,PALN metastasis |
| 2015.12.14 | Radical hysterectomy+ pelvic & paraaortic adenectomy 2016.01.14 |

TC chemo x6 course

| | |
|---|---|
| 2016.04.28 | Virchow LN metastasis |
| 2016.05.18 | AP chemo xl termination by side effect |
| 2016.07.01 | Apheresis(1) |
| 2016.07.04 | PET-CT(lt cervical LN,lt SCLN x2,lt ICLN x2,caval LN,pararectal LN) |
| 2016.07.21 | DC(08/09,11/10,11/30,12/27,2017/02/21,04/19,07/19,08/29, |

09/27,10/26,11/21,12/21,2018/01/16,02/15,05/10,08/02) 2016.07.25 IMRT

48.5Gy/10Fr/12D(lt cervical LN,lt SCLN x2,lt ICLN x2,caval

LN,pararectal LN) 2016.09.20 PET-CT Evaluation(1)

| | |
|---|---|
| | Treated sites: lt cervical LN SD,lt SCLN x2 PR,lt ICLN x2 PR,caval |
| | LN CR,pararectal LN PR |
| | New Lesion: rt iliac LN |
| 2016.10.04 | CTL(lt cervical LN,lt SCLN,rt ilac LN) |

Patient ID: 1548EM
Gender: Female
DOB: 1960/02/06

| | |
|---|---|
| 2016.11.10 | Apheresis (2), rt iliac LN PD |
| 2016.12.05 | IMRT 60.2Gy/14Fr/18D (rt iliac LN) |
| 2017.02.08 | PET-CT Evaluation (2) |
| | Treated sites: lt cerviacl LN CR, lt SCLN x2 CR, lt ICLN x2 CR, caval |
| | LN CR, pararectal LN CR, rt iliac LN PR |
| | New Lesion: liver S6 |
| 2017.03.21 | CTL (liver S6) PD |
| 2017.06.05 | RFA (liver S6) |
| 2017.07.18 | Apheresis (3) |
| 2017.09.19 | PET-CT Evaluation (3) |
| | Treated sites: rt iliac LN CR, liver S6 CR |
| | New lesion: retroduo LN |
| 2017.10.04 | Xeloda 1200mg/day |
| 2018.01.09 | PET-CT Evaluation (4) |
| | Treated sites: retroduo LN PD |
| | New lesion: none |
| 2018.01.16 | Apheresis (4) |
| | DC + Nivolumab (PD-1) 10mg (retroduo LN) |
| 2018.04.09 | PET-CT Evaluation (5) |
| | Treated sites: retroduo LN CR |
| | New lesion: none |
| 2018.07.10 | PET-CT Evaluation (6): normal study |

TABLE 4
Patient ID: 0677FS
Gender: Female
DOB: 1950/03/22
Clinical Diagnosis: Malignant lymphoma
Pathology: Follicular lymphoma
Present History:

| | |
|---|---|
| 2011.10.12 | PET-CT (lt SCLN x4, PALN x9, caval LN x8, rt renal pelvic LN x2, peritoneum x10, rt iliac |
| | LN x6, lt iliac LN x10, lt iliopsoas M, lt inguinal LN) |
| 2011.10.18 | Lt. inguinal LN biopsy: follicular lymphoma |
| 2011.10.21 | Apheresis (1) |
| 2011.10.26 | R-CHOP 5 course started, finished 2012.01.27, postponed 6^{th} course due to side effect |
| 2011.11.10 | DC (11/29, 12/20, 2012/01/19, 07/03, 2013/02/26, 03/14, 05/16, 06/10, 07/11, 08/12, |
| | 11/21, 2014/05/28, 07/02, 08/04, 09/02, 10/07, 11/04, 12/02, 2015/01/06, 02/05, |
| | 03/05, 03/31, 05/14, 08/04, 10/22, 12/17, 2016/03/08) |
| 2012.01.12 | (1) PET-CT mostly CR, remaining lesion : lt psoas muscle |
| 2012.02.09 | CTL apheresis |
| 2012.03.01 | (2) PET-CT remaining lesion : lt psoas muscle |
| 2012.03.19 | CTL (12/12, 2013/01/07, 05/17, 06/11, 07/12, 08/13, 09/10, 10/08, 2014/01/14, 05/28, |
| | 12/02, 12/17, 2015/02/05) |
| 2012.06.08 | (3) PET-CT PD:lt psoas muscle, new lesion:PALN, lt iliac LN |
| 2012.06.12 | Apheresis (2) |
| 2012.06.20 | IMRT 32.14Gy/5Fr/7D (lt psoas Mx2, PALNx2, lt iliac LNx3, rt iliac LN, lt retro- |
| | peritoneum) |

Patient ID: 0677FS

| | |
|---|---|
| 2012.07.24 | CTL apheresis |
| 2012.08.11 | (4) PET-CT Evaluation, CR all treated sites, new lesion: none |
| 2012.11.10 | (5) PET-CT Evaluation, PR lt psoas M, new lesion: rt retro-diaphragm Lig (Th11), lt iliac |
| | LNx2 |
| 2013.02.09 | (6) PET-CT Evaluation, CR lt psoas M, lt iliac LN (1), PD rt retro-diaphragm Lig. (Th11), |
| | lt iliac LN (2), new lesion: rt rib 10, Th10 (right), Th11 (right), L1 |
| 2013.02.28 | Apheresis (3) |
| 2013.03.04 | IMRT |
| 2013.04.23 | (7) PET-CT Evaluation |
| | Treated sites: rt rib10, Th10, Th11 x2, caval LN, lt iliac LN all CR |
| | New lesion: none |
| 2013.07.24 | (8) PET-CT Evaluation |
| | Treated sites: CR |
| | New lesion: lt SCLN x3 |
| 2013.10.24 | (9) PET-CT Evaluation |
| | Treated sites: lt SCLN x3 all CR |
| | New lesion: lt gluteal LN |
| 2014.01.30 | (10) PET-CT Evaluation |
| | Treated sites: lt gluteal LN PR |
| | New lesion: none |
| 2014.04.22 | (11) PET-CT Evaluation |
| | Treated sites: lt gluteal LN CR |
| | New lesion: new lt gluteal LN |
| 2014.07.22 | (12) PET-CT Evaluation |
| | Treated lesion: new lt gluteal LN CR |
| | New lesion: none |

Patient ID: 0677FS

| | |
|---|---|
| 2014.09.02 | Apheresis (4) |
| 2014.10.25 | (13) PET-CT Evaluation |
| | Treated stes: lt gluteal LN PD |
| | New lesion: lt acc LN |
| 2015.01.24 | (14) PET-CT Evaluation |
| | Treated sites: lt acc LN CR, lt gluteal LN SD |
| | New lesion: lt acc LN (2) |
| 2015.04.17 | (15) PET-CT Evaluation |
| | Treated sites: lt acc LN (2), lt gluteal LN PR |
| | New lesion: none |
| 2015.07.10 | (16) PET-CT Evaluation |
| | Treated sites: lt acc LN (2) CR, lt gluteal LN PD |
| | New lesion: lt acc LN (3), lt cervical LN, lt ICLN |
| 2015.07.15 | Apheresis (5) |
| 2015.10.09 | (17) PET-CT Evaluation |
| | Treated sites: lt cervical LN, lt acc LN (3) CR, lt ICLN, lt gluteal LN PR |
| | New lesion: none |
| 2016.01.08 | (18) PET-CT Evaluation |
| | Treated sits: lt cervical LN PR, lt gluteal LN SD, lt SCLN, lt ICLN PD |
| | New lesion: lt SCLN (2) |
| 2016.02.09 | PD-1 (lt SCLN, lt SCLN (2), lt ICLN, lt gluteal LN) |
| 2016.04.22 | (19) PET-CT Evaluation |
| | Treated sites: lt SCLN, lt SCLN (2), lt ICLN, lt gluteal LN CR |
| | New lesion: none |

TABLE 5
Patient ID: 1119HY
Gender: Male
DOB: 1948/03/22
Clinical Diagnosis: Recurrence of Lt. renal cancer post-operative
Pathology: Clear cell carcinoma
Present History:

| | |
|---|---|
| 2011.11 | Left renal cell carcinoma, Lt. nephrectomy |
| 2013.03 | Multiple lung metastasis |
| 2013.05 | Sunitinib started |
| 2014.02 | CT evaluation: PD |
| 2014.03 | Changed to Axitinib |
| 2014.05.29 | Apheresis |
| 2014.06.12 | DC (07/09, 08/05, 08/21, 09/04, 09/18, 10/02, 10/16, 11/19, 12/10, 12/24, |
| | 2015/01/15, 02/04, 02/26, 03/26, 04/23, 05/21, 06/18, 07/16, 08/10, 09/03, |
| | 10/13, 11/11, 12/10, 2016/02/10, 04/13, 05/19, 06/15, 07/12, 08/17, 09/14, 10/19, |
| | 11/17, 12/15, 2017/01/25, 02/24, 03/23, 04/24, 05/15, 06/22, 07/19, 08/23, 09/26, |
| | 2018/02/15, 04/17, 06/14, 08/22, 09/20, 10/22, 2019/01/21, 02/18, 03/11, 04/08) |
| 2014.09.19 | CT evaluation |
| | Rt. lung S3−1 PR, S3−2 PR, S5 SD, S6−1 PR, S6−2 SD, S8 PR |
| | Lt. lung S1+2 SD, S3−1 SD, S3−2 SD, S3−3 SD |
| 2015.06.10 | CT evaluation |
| | Rt. lung S3−1 SD, S3−2 CR, S5 SD, S6−1 SD, S6−2 SD, S8 PR |
| | Lt. lung S1+2 SD, S3−1 PR, S3−2 PR, S3−3 SD |
| 2015.09.18 | CT evaluation |
| | Rt. lung S3 SD, S5 PD, S6−1 PD, S6−2 PD, S8 SD |
| | Lt. lung S1+2 PD, S3−1 PD, S3−2 PD, S3−3 SD |
| 2015.10.13 | DC intra-tumoral (Rt. S5, S6−1, S6−2) |

Patient ID: 1119HY

| | |
|---|---|
| 2015.11.11 | DC intra-tumoral (Lt. S1;2, S3−1, S3−3) |
| 2016.01.13 | CT evaluation |
| | Rt. lung S3 SD, S5 SD, S6−1 PR, S6−2 PR |
| | Lt. lung S1+2 SD, S3−1 PR, S3−2 SD, S3−3 SD |
| 2016.04.08 | CT evaluation |
| | Rt. lung S3 SD, S5 PR, S6−1 PR, S6−2 SD |
| | Lt. lung S1+2 PR, S3−1 PD, S3−2 SD, S3−3 SD, S3−4 PD |
| 2016.04.13 | DC + PD-1 20mg, intra-tumoral (Lt. S3−1)...(1) |
| 2016.06.15 | CT evaluation |
| | Rt. lung S3 SD, S5 PR, S6−1 PR, S6−2 PR |
| | Lt. lung S1+2 SD, S3−1 PR, S3−2 PR, S3−3 SD, S3−4 PR |
| 2016.07.12 | DC + PD-1 10mg (Rt. S3) |
| 2016.10.01 | PET-CT evaluation |
| | Treated sites: Rt. S3 SD, Lt. S3−1 PD |
| | New lesion: None |
| 2016.10.19 | DC + PD-1 10mg (Lt. S3−1)...(2) |
| 2017.01.14 | PET-CT evaluation |
| | Treated sites: Rt. S3 PR, Lt. S3−1 PR |
| 2017.01.28 | Brain MRI: Normal study |
| 2017.02.02 | Apheresis |
| 2017.10.14 | PET-CT evaluation, acute pancreatitis |
| 2017.12.07 | ERCP, pancreatitis |
| 2018.05.18 | Apheresis |
| 2018.08.18 | PET-CT evaluation |
| | Treated sites: Rt. S3 SD, S8 PD, Lt. S1+2, S3 x2 SD |
| | New lesion: Lt. hilar LN |

Patient ID: 1119HY

| | |
|---|---|
| 2018.08.22 | DC + PD-1 20mg (Lt. hilar LN) |
| 2018.10.22 | DC + PD-1 10mg (Rt. S8) |
| 2018.12.15 | PET-CT evaluation |
| | Treated sites: Rt. S8 SD, Lt. hilar LN PR |
| | New lesion: None |
| 2019.01.21 | DC + Durvalumab (PD-L1) 25mg (Rt. S8) |
| 2019.02.18 | DC + Durvalumab (PD-L1) 50mg (Lt. hilar LN) |
| 2019.03.11 | CT evaluation |
| | Rt. lung S3 PR, S5 PR, S6 PR, S8 PR |
| | Lt. lung S1+2 SD, S3−1 CR, hilar LN PR |

TABLE 6
Patient ID: 1089KD
Gender: Female
DOB: 1958/02/14
Clinical Diagnosis: Recurrence of cervical uterine carcinoma post-operative
Pathology: Clear cell carcinoma
Present History:

| | |
|---|---|
| 2009.06 | Cervical uterine carcinoma, abdominal radical hysterectomy, Stage IIB (pT2bpN0, |
| | Clear cell carcinoma, vascular invasion ++) |
| | CDDP+CPT11 6 course |
| 2011.07.26 | Lt. iliac LN metastasis operation, Path: Clear cell carcinoma |
| 2012.09 | Lt. Int. & Ext. iliac LN metastasis operation + radiation 50.4Gy/28Fr |
| 2014.02.22 | PET-CT (lt iliac LN) |
| 2014.03.24 | Apheresis |
| 2014.04.03 | DC (04/23, 06/26, 07/23, 08/27, 10/09, 11/13, 12/04, 2015/01/15, 03/19, 04/02, |
| | 06/18, 07/16, 11/12, 2016/01/14, 02/18, 03/17, 04/14) |
| 2014.04.11 | IMRT25Gy/SF/7D(lt iliac LN) |
| 2014.06.06 | (1) PET-CT Evaluation |
| | Treated sites: lt Pelvic LN\textcircled{1} PR, lt Pelvic LN\textcircled{2}\textcircled{3} CR |
| | New lesion: none |
| 2014.06.26 | CTL (lt iliac LN\textcircled{1}) (07/23) |
| 2014.12.11 | (2) PET-CT Evaluation |
| | Treated sites: CR, New lesion: S1 |
| 2014.12.19 | CTL (S1) |
| 2015.02.19 | (3) PET-CT Evaluation |
| | Treated sites: S1 PD, New lesion: lt iliac LN x2 |
| 2015.03.23 | IMRT40Gy/SF/SD (lt S1−2, lt iliac LN) |
| 2015.04.17 | CECT: lt iliac vein embolism |
| 2015.04.30 | P53 (05/07, 05/14, 06/11, 06/18, 07/29, 09/16) |

Patient ID: 1089KD

| | |
|---|---|
| 2015.05.12 | (4) PET-CT Evaluation |
| | Treated sites: S1 CR, lt iliac LN PR~CR |
| | New lesion: lt sacrum, caval LN |
| 2015.08.27 | (5) PET-CT Evalusation |
| | Treated sites: S1 SD, caval LN PR, lt iliac LN SD, lt sacrum SD |
| | New lesion: none |
| | Pelvis MRI: lt sacral fracture |
| 2015.09.03 | Macrophage (lt iliac LN) |
| 2015.10.22 | Macrophage (S1) |
| 2015.11.27 | (6) PET-CT Evaluation |
| | Treated sites: S1 CR, caval LN CR, lt iliac LN CR, lt sacrum PR |
| | New lesion: none |
| 2016.02.05 | (7) PET-CT Evaluation |
| | Treated sites: lt sacrum CR |
| | New lesion: soft tissue close to lt sacrum |
| 2016.02.18 | DC + PD-1 10mg (soft tissue close to lt sacrum) |
| 2016.03.31 | Pelvis MRI: improvement of lt iliac vein embolism |
| 2016.05.13 | (8) PET-CT Evaluation |
| | Treated sites: soft tissue close to lt sacrum CR |
| | New lesion: none |

TABLE 7
Immune checkpoint inhibitors and matching PD-L1 assay

| | | | PDL-1 | PDL-1 | | |
|---|---|---|---|---|---|---|
| | | | IHC | antibody | | Detection |
| Drug | Company | Target | assay | Epitopes | Auto stainer | system |
| Nivolumab | BMS | PD-1 | 28-8 | Extracellular | Dako Link | Envision Flex |
| (Opdivo) | | | | | 48 | |
| Pembrolizumab | Merck | PD-1 | 22C3 | Extracellular | | |
| (Keytruda) | | | | | | |
| Atezolizumab | Roche/ | PD-L1 | SP142 | Cytoplasmic | Ventana | Optiview + |
| (Tecentrig) | Genetech | | | | Benchmark | Amplification |
| Durvalumab | Astra | PD-L1 | SP263 | Cytoplasmic | | Optiview |
| (Imfinzi) | Zeneca | | | | | |
| Avelumab | Pfizer | PD-Ll | 73-10 | Cytoplasmic | Dako Link | Envision Flex |
| (Bavencio) | | | | | 48 | |

We claim:

1. A method of regressing, reducing or eliminating tumor cells in tumor tissue of a patient comprising:
introducing intratumorally and/or through vessel a therapeutically effective amount of immature dendritic cells and/or CTLs into the patient; and
introducing intratumorally and/or through vessel and/or subcutaneously a therapeutically effective amount of PD-1 and/or PD-L1 inhibitor to the patient.

2. The method of claim 1, further including:
collecting monocyte cells and/or CTLs from the patient;
culturing the monocyte cells and/or the CTLs; and
forming immature dendritic cells from the monocyte cells.

3. The method of claim 1, wherein the introducing of the immature dendritic cells and/or CTLs, is coincident with the introducing of the PD-1 and/or PD-L1 inhibitor.

4. The method of claim 1, wherein the patient is a human.

5. The method of claim 1, wherein the introducing of the immature dendritic cells and/or CTLs is in conjunction with an adjuvant.

6. The method of claim 5, wherein the immature dendritic cells and/or CTLs and adjuvant are combined to form a composition and the composition is introduced intratumorally and/or through vessel into the patient.

7. The method of claim 5, wherein the adjuvant is selected from the group consisting of lipid-based, protein-based and polysaccharides-based adjuvants, and mixtures thereof.

8. The method of claim 7, wherein the adjuvant is selected from the group consisting of lymphocyte cultured medium, Marignase, Agaricus, OK432, BCG, Lentinan (shiitake), Reishi, Sarunokoshikake, TNF Meshimakobu, Froint's complete or incomplete adjuvant, LPS, fatty acids, TW80, phospholipids, cytokines or a virus, and mixtures thereof.

9. The method of claim 7, wherein the adjuvant comprises a leukocyte cultured medium (LCM).

10. The method of claim 9, wherein the LCM comprises at least three cytokines selected from the group consisting of eotaxin, FGF, G-CSF, GM-CSF, IFNγ, IP10, IL1β, IL1ra, IL2, IL4, IL5, IL6, IL7, IL8, IL9, IL10, IL12, IL13, IL15, IL17, MCP1, MIP1α, MIP1β, PDGFbb, RANTES, TNFα and VEGF.

11. The method of claim 1, wherein the introducing of the PD-1 and/or PD-L1 inhibitor is immediately following or a short time after the introducing of the immature dendritic cells and/or CTLs.

12. The method of claim 2, wherein culturing the monocyte cells is carried out in a culture medium selected from the group consisting of IL-4, GM-CFS, and mixtures thereof.

13. The method of claim 2, wherein culturing the CTLs is carried out in a culture medium selected from the group consisting of IL-2, CD3, and mixtures thereof.

14. The method of claim 1, wherein the tumor cells are present in metastasized tumor tissue.

15. The method of claim 11 wherein a short time after can be in a range from a couple of seconds to a couple of minutes to a couple of hours to a couple of days.

16. The method of claim 1, wherein the method is carried out in the absence of conventional therapy.

17. The method of claim 16, wherein conventional therapy is selected from chemotherapy, radiotherapy and combinations thereof.

18. The method of claim 1, further comprising introducing anti-IL-10, anti-IL-6 or mixtures thereof.

19. A method of regressing, reducing or eliminating tumor cells in a patient comprising:
obtaining monocyte cells from the patient by isolating the monocyte cells from peripheral blood mononuclear cells;
differentiating the monocyte cells to produce immature dendritic cells;
combining a first sample of the immature dendritic cells with adjuvant and Keyhole limpet to form a first mixture of the immature dendritic cells;
injecting the first mixture of the immature dendritic cells into the patient;
preparing CTLs from the monocyte-depleted peripheral blood mononuclear cells;
introducing a first sample of the CTLs into the patient subsequent to introducing the first mixture of immature dendritic cells;
combining a second sample of the immature dendritic cells with adjuvant to form a second mixture of the immature dendritic cells;
introducing the second mixture of the immature dendritic cells to the patient;
introducing a second sample of the CTLs to the patient subsequent to introducing the second mixture of the immature dendritic cells; and
introducing PD-1 and/or PD-L1 inhibitor to the patient.
